(19) United States
(12) Patent Application Publication
(10) Pub. No.: US 2014/0234937 A1
(43) Pub. Date: Aug. 21, 2014

(54) COMPOSITIONS AND METHODS FOR THE PRODUCTION OF ISOPRENE

(71) Applicants: Haitao Zhang, Chesterfield, MO (US); Maxim Suvorov, Gaithersburg, MD (US); Steven W. Hutcheson, Columbia, MD (US)

(72) Inventors: Haitao Zhang, Chesterfield, MO (US); Maxim Suvorov, Gaithersburg, MD (US); Steven W. Hutcheson, Columbia, MD (US)

(73) Assignee: AEMETIS, INC., Cupertino, CA (US)

(21) Appl. No.: 14/232,337

(22) PCT Filed: Jul. 13, 2012
(86) PCT No.: PCT/US2012/046610

§ 371 Date: Apr. 11, 2014

Related U.S. Application Data
(60) Provisional application No. 61/507,532, filed on Jul. 13, 2011.

(51) Int. Cl. C12P 5/00; C12N 15/74

(52) U.S. Cl. CPC C12P 5/007; C12N 15/74
USPC 435/167; 435/252.3; 435/471

(57) ABSTRACT

The present disclosure describes compositions and methods for production of isoprene from lignocellulosic plant biomass using a genetically engineered strain of a saprophytic bacteria.

### RELATED APPLICATIONS

This application claims priority from U.S. Provisional Patent Application No. 61/507,532, filed Jul. 13, 2011, incorporated by reference herein in its entirety.

### FIELD

The present disclosure relates to methods for production of isoprene from lignocellulosic plant biomass using a genetically engineered strain of a saprophytic bacterium.

### BACKGROUND

Isoprene is an important chemical precursor for production of rubber and some plastics. According to some estimates, around 800,000 tons of isoprene were produced in 2008. Practically all isoprene used by chemical industry is derived from petroleum, however in nature this isoprene is produced by many plants. It is estimated that plants emit around 100 billion kg of isoprene a year into the atmosphere. Despite this there are few “green” alternatives to petrochemical production of isoprene. The most promising alternative technologies utilize a number of genetically-modified microorganisms to convert organic matter into isoprene. Currently, most of these organisms can use simple sugars as a substrate for isoprene biosynthesis. The amount of such sugars in nature is very limited. Besides, the same simple sugars are also valuable for use as animal feed or human food. At the same time, according to the Department of Energy (DOE), lignocellulosic biomass is the largest sustainable carbon resource on the earth. According to the same source a billion ton of cellulosic biomass is available in the US for conversion to biofuels each year. Currently there is a need for a method of producing isoprene from lignocellulosic biomass.

### SUMMARY

In some embodiments, the disclosure provides cells in culture that produce isoprene using either mono- and di-saccharides or complex carbohydrates including but not limited to cellulose, starch, hemicelluloses and chitin. In some embodiments, the cells have a heterologous nucleic acid encoding an isoprene synthase linked to a promoter.

In some embodiments, the cells are cultured in a growth medium that includes mineral salts from sea water, an inorganic nitrogen source like ammonium chloride, peptides, proteins, vitamins and amino acids from yeast extract and tryptone, carbon sources like glucose, sucrose, lactose, starch or cellulose and hemicelluloses from corn cob.

In some embodiments, the cells contain a heterologous nucleic acid encoding an isoprene synthase and is operably linked to tac promoter. These cells start producing isoprene in the presence of isopropyl β-D-1-thiogalactopyranoside, abbreviated IPTG, in the growth medium.

In some embodiments, the cells contain a heterologous nucleic acid encoding an isoprene synthase that is operably linked to a cel9A promoter. These cells produce isoprene only in the presence of cellulose, hemicelluloses or pectins in the growth medium.

In some embodiments, the heterologous nucleic acid encoding an isoprene synthases designed to match codon usage of S. degradans and synthetically created.

### BRIEF DESCRIPTION OF THE DRAWINGS

FIG. 1 is a schematic showing the MEP/DOXP pathway. The following abbreviations are used in FIG. 1. Pur—pyruvate; G3P—glyceraldehyde 3-phosphate; DXS—1-deoxy-D-xylulose 5-phosphate synthase; DXP—1-deoxy-D-xylulose 5-phosphate; DXR—1-deoxy-D-xylulose 5-phosphate reductase; MEP—2-C-methylerythritol 4-phosphate; CMS—4-diphosphocytidyl-2-C-methyl-D-erythritol synthase; CDP-ME—4-diphosphocytidyl-2-C-methyl-D-erythritol kinase; CMK—4-diphosphocytidyl-2-C-methyl-D-erythritol kinase; CDP-MEP—2-C-methyl-D-erythritol 2,4-cyclodiphosphate; MCS—2-C-methyl-D-erythritol 2,4-cyclodiphosphate synthase; MEcPP—2-C-methyl-D-erythritol 2,4-cyclopyrophosphate; HDS—(E)-4-Hydroxy-3-methyl-but-2-enyl pyrophosphate synthase; HMB-PP—(E)-4-Hydroxy-3-methyl-but-2-enyl pyrophosphate; HDR—(E)-4-Hydroxy-3-methyl-but-2-enyl pyrophosphate reductase; IPP—isopentenyl pyrophosphate; DMAPP—dimethylallyl pyrophosphate.

FIG. 2 shows the oligonucleotide sequence of quaking aspen isoprene synthase gene optimized for expression in S. degradans (SEQ ID NO:1).

FIG. 3 shows the oligonucleotide sequence of kudzu isoprene synthase gene optimized for expression in S. degradans (SEQ ID NO:2).

FIG. 4 shows the oligonucleotide sequence of peanut isoprene synthase gene optimized for expression in S. degradans (SEQ IS NO:3).

FIG. 5 shows a schematic of plasmid pMMB503EH.

FIG. 6 shows a schematic of plasmid pZym-IPTG.

FIG. 7 is a line graph showing isoprene concentration in the overhead space of the fermenter over time.

FIG. 8 shows a sequence of the cel9A promoter area used in the cellulose-inducible construct (SEQ ID NO:4).

FIG. 9 shows sequences of primers used to amplify cel9A promoter area (SEQ ID NOs:5 (PromD) and 6 (PromR)). The MluI and EcoRI restriction endonuclease sites are shown in the lower case.

FIG. 10 shows a schematic of plasmid pZym-Cel.

### DETAILED DESCRIPTION

The present disclosure describes compositions and methods taking advantage of the natural ability of saprophytic bacteria Saccharophagus degradans (S. degradans) 2-40 to metabolize practically all complex polysaccharides found in lignocellulosic biomass. By altering the isoprenoid biosynthesis pathway in this organism, an isoprene-producing strain of S. degradans utilizing plant-derived biomass as a major carbon source was created as disclosed below.

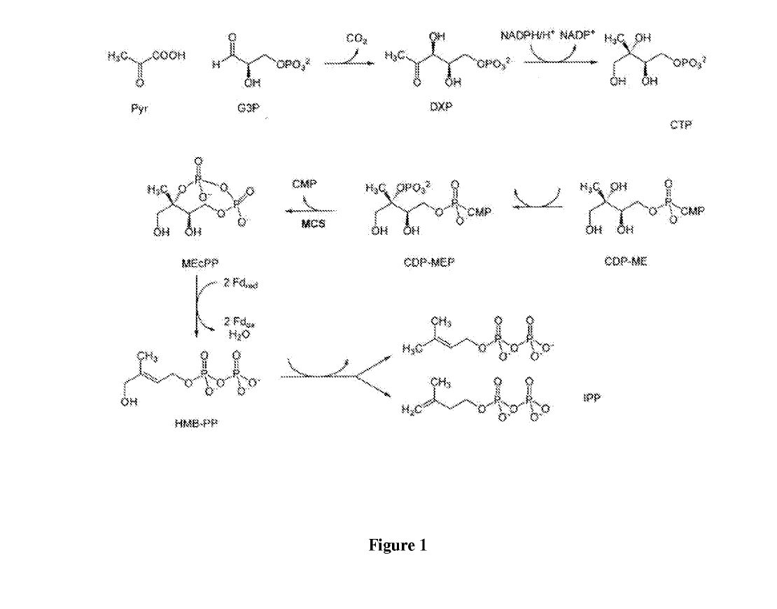

There are two major isoprenoid biosynthesis pathways utilized by living organisms: a) the mevalonate pathway or HMG-CoA reductase pathway and b) the non-mevalonate pathway or 2-C-methyl-D-erythritol 4-phosphate/1-deoxy-D-xylulose 5-phosphate pathway (MEP/DOXP pathway) (FIG. 1). Typically the mevalonate pathway present in all higher eukaryotes and many bacteria while plants and some protozoa utilize the non-mevalonate pathway.

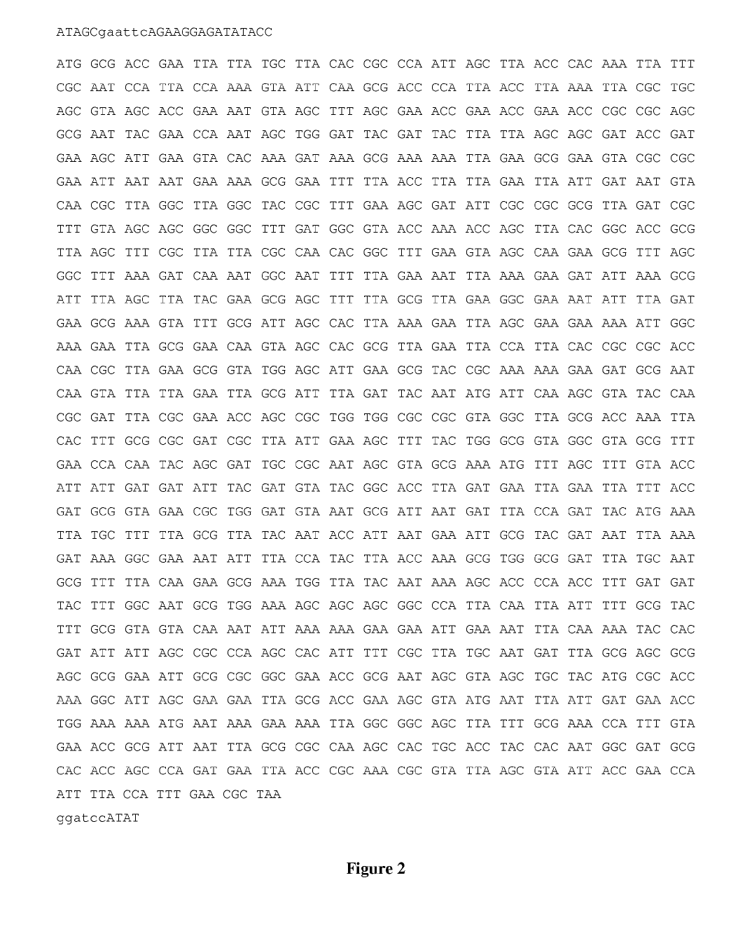

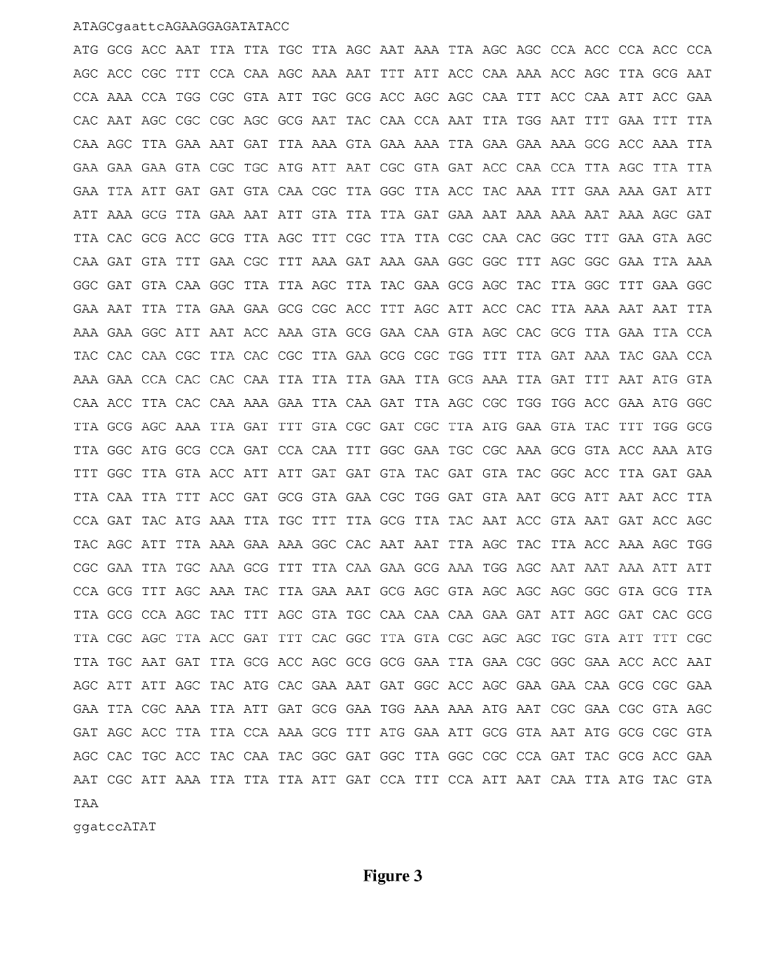

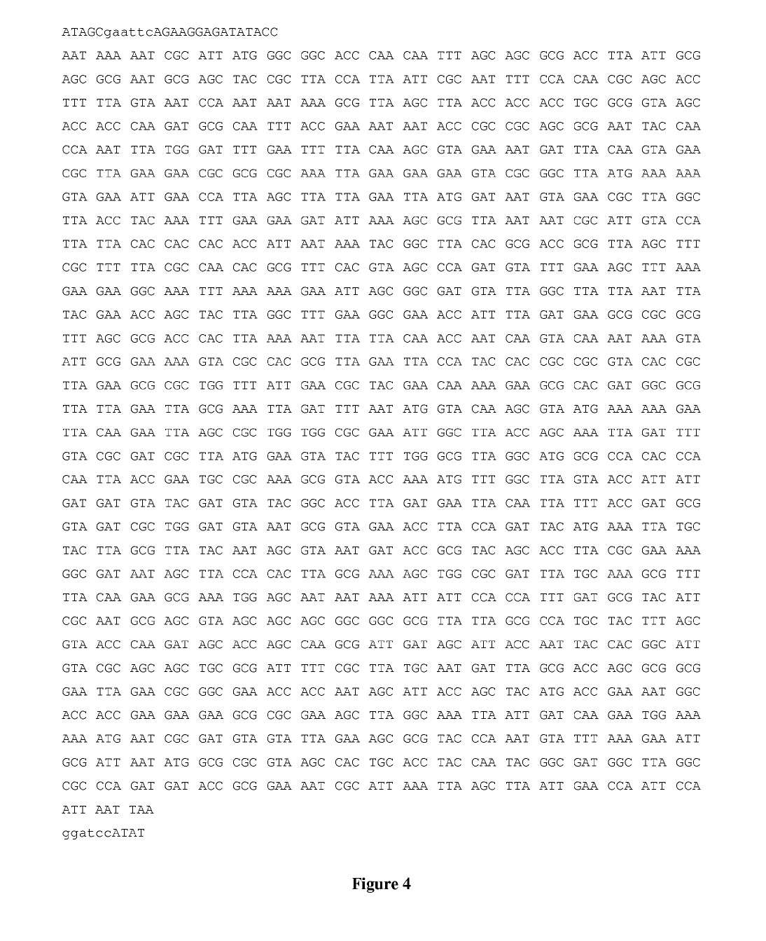

The genome of Saccharophagus degradans has been sequenced and annotated. The genome annotation clearly shows that this microorganism exclusively utilizes the non-mevalonate pathway for terpenoid biosynthesis. As was mentioned earlier, the same pathway is utilized by plants for synthesis of isoprene. The only key element missing in S. degradans for synthesis of this hydrocarbon is isoprene synthase (IspS). This enzyme converts dimethylallyl diphosphate into isoprene and diphosphate. Cells of many higher plants (tracheophytes) contain isoprene synthases. Amino acid sequences for quaking aspen, kudzu and peanut isoprene syntheses were obtained from Gene Bank. Based on these sequences, oligonucleotide sequences for all three isoprene synthases were designed. The oligonucleotide sequences were designed to match the codon usage table of 10 highest expressed genes in S. degradans. (FIGS. 2-4) The oligonucleotide sequences encoding the aforementioned isoprene synthases were synthesized with EcoRI endonuclease site followed by the ribosome-biding site upstream from the start-codon of the IspS-encoding sequences and with BamHI endonuclease site following the stop-codon of the gene. The synthetic isoprene synthase genes were lacking the signal-peptide-encoding region present in the wild-type versions of the genes.

In certain embodiments, any heterologous nucleic acid encoding an isoprene synthase can be introduced into any saprophytic bacteria, including S. degradans. Moreover, any variant of a heterologous nucleic acid encoding an isoprene synthase can be introduced into any saprophytic bacteria including S. degradans. Isoprene synthase amino acid sequences include Arachis hypogaea (peanut) isoprene synthase (GenBank accession number: EZ721087.1); Populus tremuloides (aspen) isoprene synthase (GenBank accession number: AAQ16588); Pueraria lobata (Kudzu vine) (Pueraria montana var. lobata) isoprene synthase (Uniprot accession number: UPI00003B9580).

Any variant of these amino acid sequences may be introduced into a saprophytic bacterium, including S. degradans. These variants may have between 70-99% sequence identity with any wild type isoprene synthase, as long as they have the ability to synthesize isoprene.

The heterologous nucleic acid encoding an isoprene synthase can include any promoter that allows for the expression of isoprene synthase. This includes promoters from S. degradans genes. These genes include any of the ones described in U.S. Patent Publication No. 2008-0293115, incorporated herein by reference in its entirety.

Promoters from other organisms can also be used. These promoters include the tac promoter and the LacUV5 promoter. The LacUV5 promoter can be used in a triple configuration.

DNA sequences encoding isoprene synthase may be cloned into any suitable vectors for expression in intact saprophytic bacteria or in cell-free translation systems by methods well-known in the art.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that 1) confer resistance to antibiotics or other toxic substances, e.g., ampicillin, neomycin, methotrexate, etc.; 2) complement auxotrophic deficiencies, or 3) supply critical nutrients not available from complex media, e.g., the gene encoding D-alanine racemase for Bacilli. Markers may be an inducible or non-inducible gene and will generally allow for positive selection. Non-limiting examples of markers include the ampicillin resistance marker (i.e., β-lactamase), tetracycline resistance marker, neomycin/kanamycin resistance marker (i.e., neomycin phosphotransferase), dihydrofolate reductase, glutamine synthetase, and the like. The choice of the proper selectable marker will depend on the saprophytic bacteria, and appropriate markers for different saprophytic bacteria as understood by those of skill in the art.

Vectors can contain one or more replication and inheritance systems for cloning or expression, one or more markers for selection in the saprophytic bacteria, e.g., antibiotic resistance, and one or more expression cassettes. The inserted coding sequences can be synthesized by standard methods, isolated from natural sources, or prepared as hybrids. Ligation of the coding sequences to transcriptional regulatory elements (e.g., promoters, enhancers, and/or insulators) and/or to other amino acid encoding sequences can be carried out using established methods.

Expression vectors for saprophytic bacteria ordinarily include an origin of replication (where extrachromosomal amplification is desired, as in cloning, the origin will be a bacterial origin), a promoter located upstream from the isoprene synthase coding sequences, together with a ribosome binding site (the ribosome binding or Shine-Dalgarno sequence is only needed for prokaryotic expression), RNA splice site (if the isoprene synthase DNA contains genomic DNA containing one or more introns), a polyadenylation site, and a transcriptional termination sequence. As noted, the skilled artisan will appreciate that certain of these sequences are not required for expression in certain saprophytic bacteria. An expression vector for use with microbes need only contain an origin of replication recognized by the intended saprophytic bacteria, a promoter which will function in the host and a phenotypic selection gene, for example a gene encoding proteins conferring antibiotic resistance or supplying an auxotrophic requirement.

Saprophytic bacteria can be transformed, transfected, or infected as appropriate by any suitable method including electroporation, calcium chloride-, lithium chloride-, lithium acetate/polyethylene glycol-, calcium phosphate-, DEAE-dextran-, liposome-mediated DNA uptake, spheroplasting, injection, microinjection, microprojectile bombardment, phage infection, viral infection, or other established methods. Vectors may integrate into the saprophytic bacteria genome or remain separate from the host cell genome. Alternatively, vectors containing the nucleic acids of interest can be transcribed in vitro, and the resulting RNA introduced into the saprophytic bacteria by well-known methods, e.g., by injection. The cells into which have been introduced nucleic acids described above are meant to also include the progeny of such cells. Methods of transfection include nucleofection, electroporation, sonoporation, heat shock, magnetofection and proprietary transfection reagents such as Lipofectamine, Dojindo, GenePORTER, Hilymax, Fugene, jetPEI, Effectene or DreamFect.

Saprophytic bacteria carrying an expression vector (i.e., transformants or clones) are selected using markers depending on the mode of the vector construction. The marker may be on the same or a different DNA molecule, preferably the same DNA molecule.

In prokaryotic hosts, the transformant may be selected, e.g., by resistance to ampicillin, tetracycline or other antibiotics. Production of a particular product based on temperature sensitivity may also serve as an appropriate marker.

### DEFINITIONS

As used herein, the terms “transformed”, “stably transformed” or “transgenic” with reference to a cell means the cell has a non-native (heterologous) nucleic acid sequence integrated into its genome or as an episomal plasmid that is maintained through multiple generations.

As used herein, the term “expression” refers to the process by which a polypeptide is produced based on the nucleic acid sequence of a gene. The process includes both transcription and translation.

The term “introduced” in the context of inserting a nucleic acid sequence into a cell, means “transfection”, or “transformation” or “transduction” and includes reference to the incorporation of a nucleic acid sequence into a eukaryotic or prokaryotic cell where the nucleic acid sequence may be incorporated into the genome of the cell (for example, chromosome, plasmid, plastid, or mitochondrial DNA), converted into an autonomous replicon, or transiently expressed (for example, transfected mRNA).

“A”, “an” and “the” include plural references unless the context clearly dictates otherwise.

Numeric ranges are inclusive of the numbers defining the range.

The term “variant” refers to a protein or polypeptide in which one or more amino acid substitutions, deletions, and/or insertions are present as compared to the amino acid sequence of an protein or peptide and includes naturally occurring allelic variants or alternative splice variants of an protein or peptide. The term “variant” includes the replacement of one or more amino acids in a peptide sequence with a similar or homologous amino acid(s) or a dissimilar amino acid(s). There are many scales on which amino acids can be ranked as similar or homologous. (Gunnar von Heijne, Sequence Analysis in Molecular Biology, p. 123-39 (Academic Press, New York, N.Y. 1987.) Preferred variants include alanine substitutions at one or more of amino acid positions. Other preferred substitutions include conservative substitutions that have little or no effect on the overall net charge, polarity, or hydrophobicity of the protein. Conservative substitutions are set forth in the table below. According to some embodiments, the SP1NT1 and TMPRSS4 polypeptides have at least 80%, 85%, 88%, 95%, 96%, 97%, 98% or 99% sequence identity with the amino acid sequences of the preferred embodiments.

### Conservative Amino Acid Substitutions

| | |
|---|---|
| Basic: | arginine |
| | lysine |
| | histidine |
| Acidic: | glutamic acid |
| | aspartic acid |
| Uncharged Polar: | glutamine |
| | asparagines |
| | serine |
| | threonine |
| | tyrosine |
| Non-Polar: | phenylalanine |
| | tryptophan |
| | cysteine |
| | glycine |
| | alanine |
| | valine |
| | praline |
| | methionine |
| | leucine |
| | isoleucine |

The table below sets out another scheme of amino acid substitution:

| Original | |
|---|---|
| Residue | Substitutions |
| Ala | Gly; Ser |
| Arg | Lys |
| Asn | Gln; His |
| Asp | Glu |
| Cys | Ser |
| Gln | Asn |
| Glu | Asp |
| Gly | Ala; Pro |
| His | Asn; Gln |
| Ile | Leu; Val |
| Leu | Ile; Val |
| Lys | Arg; Gln; Glu |
| Met | Leu; Tyr; Ile |
| Phe | Met; Leu; Tyr |
| Ser | Thr |
| Thr | Ser |
| Trp | Tyr |
| Tyr | Trp; Phe |
| Val | Ile; Leu |

Other variants can consist of less conservative amino acid substitutions, such as selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions that in general are expected to have a more significant effect on function are those in which (a) glycine and/or proline is substituted by another amino acid or is deleted or inserted; (b) a hydrophilic residue, e.g., seryl or threonyl, is substituted for (or by) a hydrophobic residue, e.g., leucyl, isoleucyl, phenylalanyl, valyl, or alanyl; (c) a cysteine residue is substituted for (or by) any other residue; (d) a residue having an electropositive side chain, e.g., lysyl, arginyl, or histidyl, is substituted for (or by) a residue having an electronegative charge, e.g., glutamyl or aspartyl; or (e) a residue having a bulky side chain, e.g., phenylalanine, is substituted for (or by) one not having such a side chain, e.g., glycine. Other variants include those designed to either generate a novel glycosylation and/or phosphorylation site(s), or those designed to delete an existing glycosylation and/or phosphorylation site(s). Variants include at least one amino acid substitution at a glycosylation site, a proteolytic cleavage site and/or a cysteine residue. Variants also include proteins and peptides with additional amino acid residues before or after the protein or peptide amino acid sequence on linker peptides. The term “variant” also encompasses polypeptides that have the amino acid sequence of the proteins/peptides of the present invention with at least one and up to 25 (e.g., 5, 10, 15, 20) or more (e.g., 30, 40, 50, 100) additional amino acids flanking either the 3′ or 5′ end of the amino acid sequence.

The headings provided herein are not limitations of the various aspects or embodiments of the invention, which can be had by reference to the specification as a whole.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

### EXAMPLES

The following examples further support, but do not exclusively represent, preferred embodiments of the present invention.

### Example 1

### IPTG-Inducible Strain (Zym-IPTG)

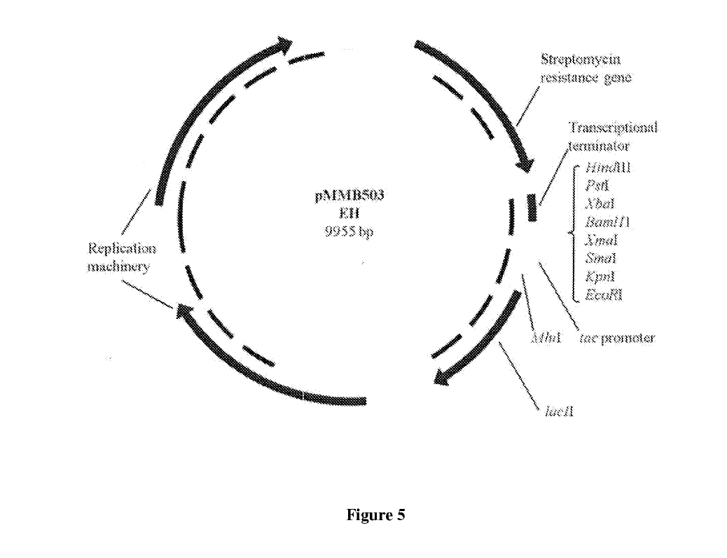

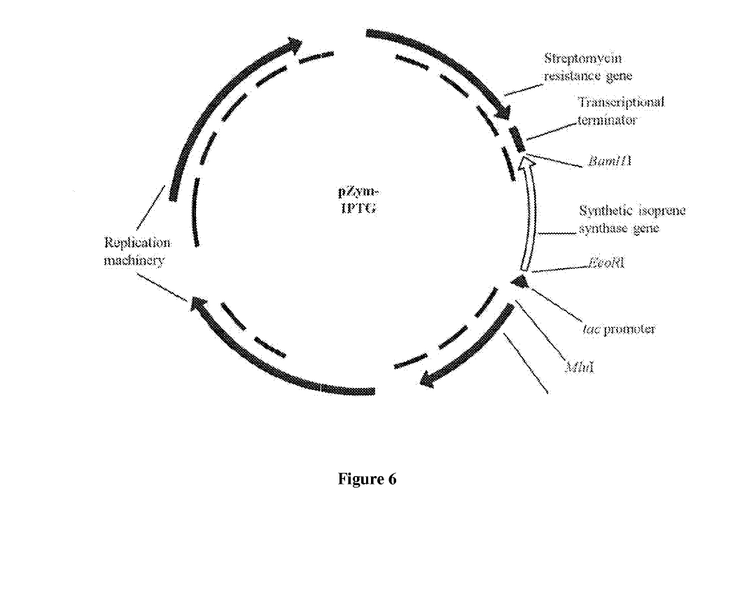

The resulting synthetic oligonucleotide sequences were digested with EcoRI and BamHI endonucleases and cloned into EcoRI and BamHI sites of pMMB503EH plasmid (FIG. 5). The resulting plasmids containing isoprene synthase genes (FIG. 6) were electroporated into S. degradans strain 2-40. Selection of the cells containing the plasmid was performed on agar supplemented with 50 μg/mL streptomycin. In the resulting construct the IspS-encoding sequences were under transcriptional regulation of the tac promoter. The S. degradans strains expressing the isoprene synthases were cultivated in 250 mL shake flasks at 30° C. Expression of the isoprene synthases in the described strains was induced by adding 0.4 mM isopropyl β-D-1-thiogalactopyranoside (IPTG) to the growth medium in the middle of the log-phase. Detection and quantification of the isoprene in the flasks was performed using RAE-106 volatile organic compound detector. Isoprene in the overhead space of the shake flasks also manifested itself in “refinery” smell.

For more accurate assessment of isoprene production capabilities of the described strains 10 L Bioflo fermenters were used. Cultivation of the bacterial cells was performed at 30° C. Composition of the 2×2-40 growth medium used for that was as follows:

Instant Ocean sea salt—230 g
Yeast extract—40 g
Casein peptone—50 g
Ammonium chloride—10 g

### Glucose—100 g

### Water—to 10 L

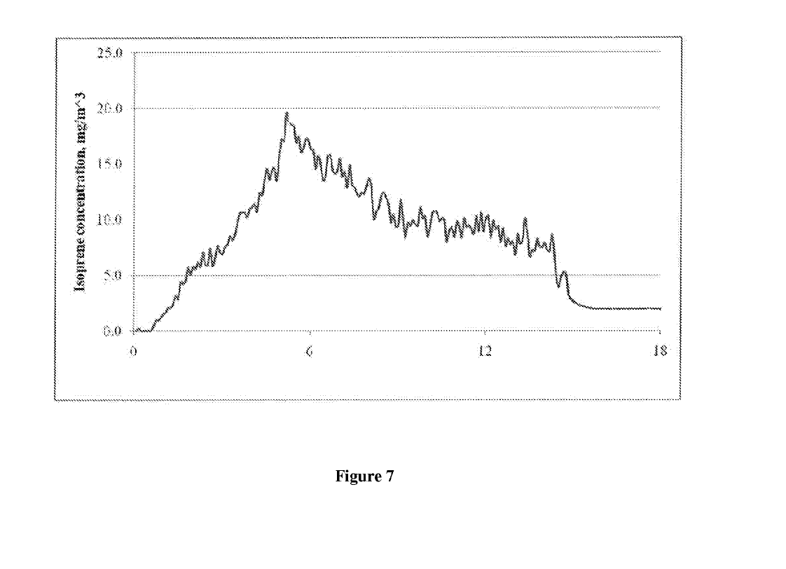

Concentration of isoprene in the overhead space of the fermenters was constantly monitored using RAE-106 detectors (FIG. 7). As an alternative to glucose, 1% (w/v) pretreated corn cob was also used as a major carbon source for isoprene production.

### Example 2

### The Cellulose-Inducible Strain (Zym-Cel)

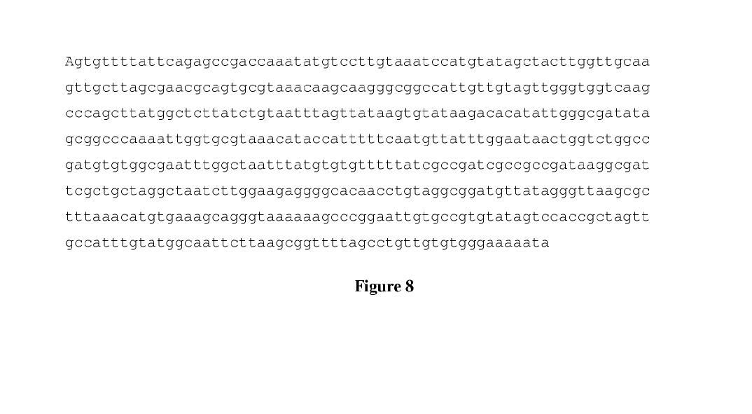

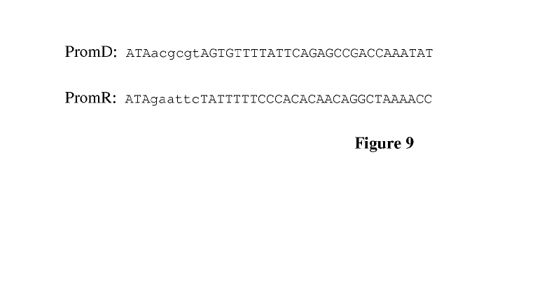

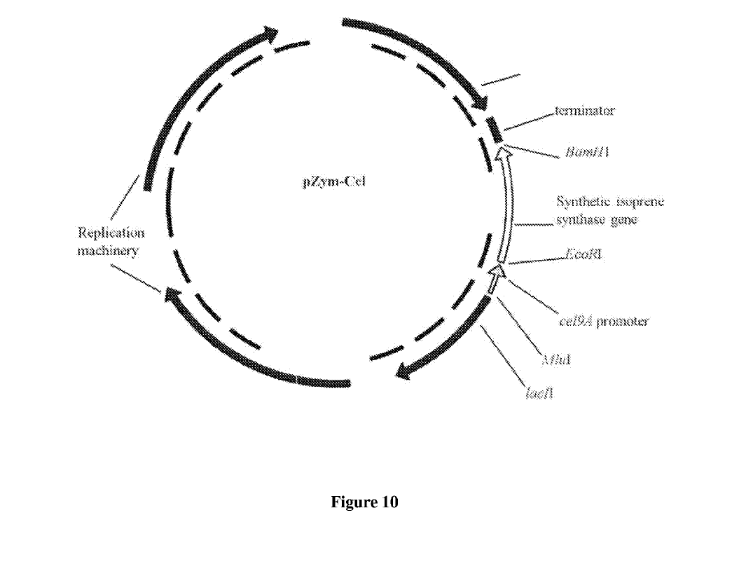

In order to reduce cost of the isoprene production using S. degradans-based strain, a novel cellulose-inducible expression system was created. The key element of this system is promoter of cel9A gene encoding an endo-1,4-β-glucanase in S. degradans. Using qRT-PCR and deep sequencing techniques, cel9A gene was identified as the highest transcribed gene among other carbohydrase-encoding genes in S. degradans. Transcription of this gene is highly upregulated in the presence of any cellulose and/or hemicelluloses-containing material in the growth medium. In order to create the cellulose-inducible protein expression system for S. degradans, the 500-bp-long nucleotide sequence (FIG. 8) upstream from cel9A was PCR-amplified. The resulting PCR fragment (FIG. 9) contained cel9A promoter, operator regions as well as ribosome-binding site. The PCR primers contained MluI and EcoRI restriction endonuclease sites. The resulting PCR fragment was digested with MluI and EcoRI restriction endonucleases and cloned into MluI and EcoRI sites of pMMB503EH plasmid (FIG. 5). Then the isoprene synthase genes were cloned into EcoRI and BamHI sites of the resulting plasmid (FIG. 10).

1. A saprophytic bacteria comprising an isoprene synthase.

2. The saprophytic bacteria of claim 1, wherein the saprophytic bacteria is Saccharophagus degradans 2-40.

3. The saprophytic bacteria of claim 1, wherein the isoprene synthase is selected from the group consisting of quaking aspen isoprene synthase, kudzu isoprene synthase and peanut isoprene synthase.

4. The saprophytic bacteria of claim 1, wherein the saprophytic bacteria comprises a heterologous nucleic acid encoding isoprene synthase.

5. The saprophytic bacteria of claim 4, wherein the heterologous nucleic acid is operably linked to a promoter selected from a tac promoter and a cel9A promoter.

6. The saprophytic bacteria of claim 4, wherein the heterologous nucleic acid matches codon usage of Saccharophagus degradans 2-40.

7. A method of producing isoprene comprising
(a) providing a saprophytic bacteria comprising an isoprene synthase; and
(b) culturing the saprophytic bacteria in a media comprising a carbon source, thereby producing isoprene.

8. The method of claim 7, wherein the saprophytic bacteria is Saccharophagus degradans 2-40.

9. The method of claim 8, wherein the growth media further comprises mineral salts from sea water.

10. The method of claim 7, wherein the growth media further comprises an inorganic nitrogen source.

11. The method of claim 7, wherein the carbon source is selected from the group consisting of glucose, sucrose, lactose, starch, cellulose, hemicellulose and corn cob.

12. The method of claim 7, wherein the isoprene synthase is selected from the group consisting of quaking aspen isoprene synthase, kudzu isoprene synthase and peanut isoprene synthase.

13. The method of claim 7, wherein the saprophytic bacteria comprises a heterologous nucleic acid encoding isoprene synthase.

14. The method of claim 13, wherein the heterologous nucleic acid is operably linked to a promoter selected from a tac promoter and a cel9A promoter.

15. The method of claim 8, wherein the heterologous nucleic acid matches codon usage of Saccharophagus degradans 2-40.

16. A method of producing a bacteria that produces isoprene in the presence of a carbon source comprising
(a) providing a saprophytic bacteria; and
(b) introducing to the saprophytic bacteria a heterologous nucleic acid encoding an isoprene synthase,
thereby producing a bacteria that produces isoprene in the presence of a carbon source.

17. The method of claim 16, wherein the saprophytic bacteria is Saccharophagus degradans 2-40.

18. The method of claim 16, wherein the carbon source is selected from the group consisting of glucose, sucrose, lactose, starch, cellulose, hemicellulose and corn cob.

19. The method of claim 16, wherein the isoprene synthase is selected from the group consisting of quaking aspen isoprene synthase, kudzu isoprene synthase and peanut isoprene synthase.

20. The method of claim 17, wherein the heterologous nucleic acid is operably linked to a promoter selected from a tac promoter and a cel9A promoter.

21. The method of claim 17, wherein the heterologous nucleic acid matches codon usage of Saccharophagus degradans 2-40.
